(19) United States
(12) Patent Application Publication
(10) Pub. No.: US 2021/0106235 A1
(43) Pub. Date: Apr. 15, 2021

(54) DEVICE AND METHOD FOR DETECTING COUPLING BETWEEN PHYSIOLOGICAL SIGNALS

(71) Applicants: Han-Ping HUANG, New Taipei City (TW); Crystalvue Medical Corporation, Taoyuan City 330 (TW)

(72) Inventor: Han-Ping HUANG, Hsinchu City (TW)

(21) Appl. No.: 17/065,511

(22) Filed: Oct. 7, 2020

Related U.S. Application Data
(60) Provisional application No. 62/913,004, filed on Oct. 9, 2019.

(51) Int. Cl. A61B 5/0205; A61B 5/00

(52) U.S. Cl. CPC A61B 5/0205; A61B 5/6831; A61B 5/7246; A61B 5/7253; A61B 5/6823

(57) ABSTRACT

A device for detecting coupling between physiological signals includes a first signal acquisition unit, a second signal acquisition unit and an operation unit. The first signal acquisition unit is used to acquire a first physiological signal of an examinee. The second signal acquisition unit is used to acquire a second physiological signal of the examinee. The second physiological signal is different from the first physiological signal. The operation unit is coupled to the first signal acquisition unit and the second signal acquisition unit respectively to quantify a matching state of the first physiological signal and/or the second physiological signal at different time points as a specific index to indicate whether a physiological state of the examinee is normal.

### CROSS REFERENCE TO RELATED APPLICATIONS

This Application is a non-provisional application claiming priority to U.S. Provisional Application 62/913,004 filed on Oct. 9, 2019, which is incorporated by reference in its entirety.

### BACKGROUND OF THE INVENTION

### Field of the Invention

The invention relates to physiological signal detection; in particular, to a device and a method for detecting coupling between physiological signals.

### Description of the Prior Art

In recent years, five of the ten leading causes of death in Taiwan are related to cardiovascular disease, and cardiovascular disease ranks second. In terms of cardiovascular disease alone, the mortality rate is about 10%, but if the closely related cerebrovascular diseases and diabetes, which are prone to cardiovascular complications, are added, the total mortality rate is even as high as 25%. In other words, the risk of cardiovascular disease is no less than that of cancer, which is the top ten cause of death. Therefore, “cardiovascular examination” is an important examination item for assessing whether the cardiovascular function is normal or not.

Generally speaking, “cardiovascular examination” is mainly divided into two types: non-invasive and invasive. For more common and convenient non-invasive cardiovascular examinations, exercise treadmill test is usually used to allow examinees to run on a treadmill to increase cardiac oxygen consumption and physical load. During exercise, the examinee's heart is hypoxic due to coronary artery obstruction, and there will be a significant decrease in ST segment on the electrocardiogram, so as to infer whether the examinee has coronary artery disease.

Although many current medical guidelines recommend that patients use the above-mentioned non-invasive exercise electrocardiogram test for cardiovascular disease assessment, the practical application of this method has troubled clinicians. The reason is that although this method has good sensitivity (79%) and specificity (80%), it is still clinically in the majority of healthy examinees. If the specificity is not 100%, it is easy to cause exercise ECG A large part of the positive results after the test are false positives. Therefore, it is usually necessary to further use invasive cardiovascular examinations (such as coronary angiography and other imaging examinations) to confirm those false positive results that cannot be confirmed by exercise electrocardiography. This not only reduces the doctor's confidence in the exercise electrocardiogram to judge cardiovascular diseases, but also wastes a lot of medical resources in disguise.

Therefore, there is still a lack of a coronary artery disease detection device that can provide more accurate detection results. However, due to the non-invasive characteristics of exercise electrocardiogram and the convenience of wide application, it is difficult to be completely replaced. Thus, how to improve the clinical accuracy of exercise electrocardiogram for the evaluation of cardiovascular diseases in order to effectively solve the clinical problem of low positive accuracy rate because healthy examinees are in the majority is an urgent issue that needs to be solved.

### SUMMARY OF THE INVENTION

Therefore, the invention provides to a device and a method for detecting coupling between physiological signals to solve the above-mentioned problems of the prior arts.

A preferred embodiment of the invention is a device for detecting coupling between physiological signals. In this embodiment, the device for detecting coupling between physiological signals includes a first signal acquisition unit, a second signal acquisition unit and an operation unit. The first signal acquisition unit is used to acquire a first physiological signal of an examinee. The second signal acquisition unit is used to acquire a second physiological signal of the examinee. The second physiological signal is different from the first physiological signal. The operation unit is coupled to the first signal acquisition unit and the second signal acquisition unit respectively to quantify a matching state of the first physiological signal and/or the second physiological signal at different time points as a specific index to indicate whether a physiological state of the examinee is normal.

In an embodiment, the first signal acquisition unit and the second signal acquisition unit acquire the first physiological signal and the second physiological signal of the examinee respectively when the examinee is in an exercise state.

In an embodiment, the operation unit uses Hilbert-Huang transform (HHT) to convert the matching state to the specific index.

In an embodiment, the specific index is used to determine whether the examinee has a specific disease.

In an embodiment, the specific disease is a coronary artery disease.

In an embodiment, the first physiological signal and the second physiological signal are related to a heart rhythm information and a breathing information of the examinee respectively.

In an embodiment, the first signal acquisition unit acquires the heart rhythm information of the examinee through electrocardiography (ECG) or photoplethysmography (PPG).

In an embodiment, the second signal acquisition unit acquires the breathing information of the examinee through a chest strap, a nasal flow detector or an electrocardiography derived respiration (EDR) algorithm.

In an embodiment, the device is a non-invasive detection device.

Another preferred embodiment of the invention is a method for detecting coupling between physiological signals, comprising steps of: (a) acquiring a first physiological signal of an examinee; (b) acquiring a second physiological signal of the examinee, wherein the second physiological signal is different from the first physiological signal; (c) calculating a matching state of the first physiological signal and/or the second physiological signal at different time points; and (d) quantifying the matching state as a specific index to indicate whether a physiological state of the examinee is normal.

Compared to the prior art, the device and method for detecting the coupling of physiological signals of the invention are non-invasive and can quickly determine whether the examinee has cardiovascular disease by collecting the heart rhythm and breathing data of the examinee during exercise, thereby effectively improving the disease determination accuracy of the exercise ECG on cardiovascular disease, so there is no need to further confirm the false positive test results of the exercise ECG, to achieve the substantial effect of saving medical resources and improving medical efficiency and quality.

The advantage and spirit of the invention may be understood by the following detailed descriptions together with the appended drawings.

### BRIEF DESCRIPTION OF THE APPENDED DRAWINGS

FIG. 1 illustrates a schematic diagram of a device for detecting the coupling of physiological signals according to a preferred embodiment of the invention.

FIG. 2 illustrates a flowchart of a method for detecting the coupling of physiological signals according to another preferred embodiment of the invention.

FIG. 3 illustrates a schematic diagram showing higher coupling between the heart rhythm signal and the breathing signal of the examinee during exercise.

FIG. 4 illustrates a schematic diagram showing lower coupling between the heart rhythm signal and the breathing signal of the examinee during exercise.

FIG. 5 illustrates a schematic diagram showing the index corresponding to the coupling between the heart rhythm signal and the breathing signal of a healthy examinee during exercise under two different modes (frequency) changing with time (stage).

FIG. 6 illustrates a schematic diagram showing the index corresponding to the coupling between the heart rhythm signal and the breathing signal of an examinee with coronary artery disease during exercise under two different modes (frequency) changing with time (stage).

### DETAILED DESCRIPTION OF THE INVENTION

Exemplary embodiments of the invention are referenced in detail now, and examples of the exemplary embodiments are illustrated in the drawings. Further, the same or similar reference numerals of the components/components in the drawings and the detailed description of the invention are used on behalf of the same or similar parts.

The invention provides a device and method for detecting the coupling of physiological signals, which are used to detect the coupling of at least one physiological signal of an examinee at different time points (such as before and after the detection process) in a non-invasive manner. The method can first collect the time series of at least one physiological signal of the examinee, such as the heart rhythm sequence and/or the respiratory sequence, and then decompose the sequence to calculate the coupling of the mode corresponding to the at least one physiological signal at different time points, and then determine whether the state of the examinee is normal according to the change of the coupling.

In the process of exercise ECG detection, the device and method for detecting the coupling of physiological signals of the invention will first collect two physiological signals (heart rhythm data and respiratory data) of the examinee during exercise, and then perform mode decomposition on these two physiological signals (heart rhythm data and breathing data) and extract suitable modes corresponding to different exercise stages according to the corresponding average frequency, and then compare the phase matching of the same suitable modalities of these two physiological signals (heart rhythm data and breathing data) during exercise, and then Hilbert-Huang transform (HHT) is used to convert the phase matching into an index to indicate whether the state of the examinee during exercise is normal (whether suffering from cardiovascular disease), but not limited to this. In fact, in addition to the coupling between the above-mentioned two physiological signals, it can also be the coupling of the two physiological signals before and after measurement (for example, waiting before exercise and rest after exercise, but not limited to this) in the invention; in addition to the above-mentioned phase matching method, it can also be the matching of other indexes and characteristics in the invention; in addition to the above-mentioned method of converting the matching into an index through Hilbert-Huang transform (HHT), it can also other methods for conversion in the invention.

It should be noted that, the human body will centrally regulate the heart rhythm and respiration by the positive and parasympathetic nerves under the normal condition. For example, inhalation will make the heart rhythm faster, while exhalation will slow it down. However, for patients with cardiovascular diseases, coronary artery disease will cause cardiac ischemia during exercise, which will affect the above-mentioned regulation relationship. This is the key that the detection device and method for detecting the coupling of the physiological signals of the invention can determine whether the examinee has coronary artery disease during exercise.

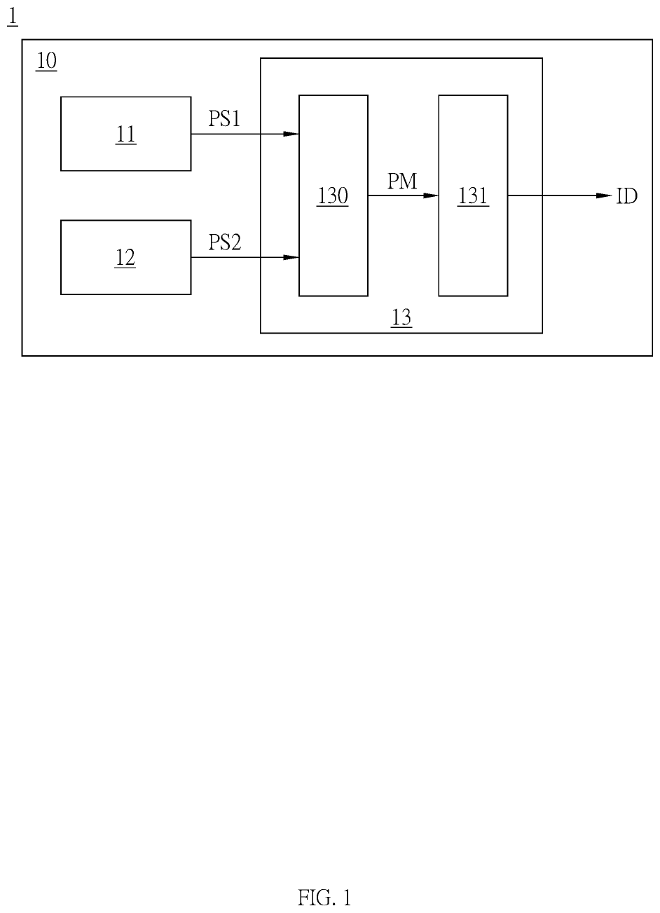

An embodiment of the invention is a non-invasive detection device for detecting the coupling of the physiological signals. Please refer to FIG. 1. FIG. 1 illustrates a schematic diagram of the detection device for detecting the coupling of the physiological signals in this embodiment.

As shown in FIG. 1, the detection device 1 includes a device body 10, a first signal acquisition unit 11, a second signal acquisition unit 12 and an operation unit 13. The first signal acquisition unit 11 is disposed on the device body 10 to acquire the first physiological signal PS1 of the examinee during exercise. The second signal acquisition unit 12 is disposed on the device body 10 to acquire the second physiological signal PS2 of the examinee during exercise.

It should be noted that the second physiological signal PS2 is different from the first physiological signal PS1. For example, the first physiological signal PS1 and the second physiological signal PS2 can be related to the heart rhythm information and breathing information of the examinee during exercise respectively, but not limited to this. In addition, instead of being disposed on the main body 10 of the device, the first signal acquisition unit 11 and the second signal acquisition unit 12 can be connected to the operation unit 13 in a wireless or wired manner to transmit the first physiological signal PS1 and the second physiological signal PS2 to the operation unit 13.

In practical applications, the first signal acquisition unit 11 can use electrocardiogram (ECG) or photoplethysmogram (PPG) to acquire the heart rhythm information of the examinee during exercise, but not limited to this. The second signal acquisition unit 12 can use a chest strap, a nasal flow detector or an Electrocardiography Derived Respiration (EDR) algorithm to acquire the breathing information of the examinee during exercise, but not limited to this.

The operation unit 13 is disposed on the device body 10 and is coupled to the first signal acquirement unit 11 and the second signal acquirement unit 12 respectively. After the first signal acquirement unit 11 and the second signal acquirement unit 12 acquire the first physiological signal PS1 and the second physiological signal PS2 of the examinee respectively, the operation unit 13 can simultaneously receive the first physiological signal PS1 and the second physiological signal PS2 from the first signal acquirement unit 11 and the second signal acquirement unit 12.

The operation unit 13 can include a first processing unit 130 and a second processing unit 131. The second processing unit 131 is coupled to the first processing unit 130. The first processing unit 130 calculates the matching state PM of the first physiological signal PS1 and/or the second physiological signal PS2 according to an algorithm, and then the second processing unit 131 quantifies the matching state PM into a specific index ID to indicate whether the physiological state of the examinee is normal.

In practical applications, the first signal acquisition unit 11 and the second signal acquisition unit 12 can acquire the first physiological signal PS1 and the second physiological signal PS2 respectively when the examinee is in an exercise state; the second processing unit 131 in the operation unit 13 can use Hilbert-Huang transform (HHT) or other conversion methods to convert the matching state PM (for example, the matching state of phase or other index/characteristics) of the first physiological signal PS1 and/or the second physiological signal PS2 at different time points (for example, before and after the measurement) into a specific index ID, but not limited to this.

It should be noted that the quality of the phase matching or the matching state PM of other indexes and characteristics between the first physiological signal PS1 and the second physiological signal PS2 is related to the coupling between the first physiological signal PS1 and the second physiological signal PS2, but not limited to this.

For example, when the coupling between the first physiological signal PS1 and the second physiological signal PS2 is high, it means that the matching state PM of the phase or other indexes and characteristics between the first physiological signal PS1 and the second physiological signal PS2 is better. Conversely, when the coupling between the first physiological signal PS1 and the second physiological signal PS2 is low, it means that the matching state PM of the phase or other indexes and characteristics between the first physiological signal PS1 and the second physiological signal PS2 is poor.

It should be noted that the quality of the matching state PM of the phase or other indexes and characteristics between the first physiological signal PS1 and the second physiological signal PS2 before and after measurement (such as waiting before exercise and rest after exercise, but not limited to this) is related to the coupling between the first physiological signal PS1 and the second physiological signal PS2 before and after the measurement, but not limited to this.

For example, when the first physiological signal PS1 and the second physiological signal PS2 have high coupling between before and after the measurement, it means that the matching state PM of the phase or other indexes and characteristics of the first physiological signal PS1 and the second physiological signal PS2 before and after the measurement is better. Conversely, when the coupling between the first physiological signal PS1 and the second physiological signal PS2 before and after the measurement is low, it means that the matching state PM of the phase or other indexes and characteristics of the first physiological signal PS1 and the second physiological signal PS2 before and after the measurement is poor.

In another embodiment, it is assumed that the first physiological signal PS1 is heartbeat data and it has indexes and characteristics of related HRV parameters. At this time, it can determine the quality of the matching state PM of the first physiological signal PS1 before and after exercise based on whether the HRV parameter of the measured heartbeat data is lower than a threshold value before and after exercise. If the HRV parameter of the heartbeat data of the examinee is lower than the threshold before and after exercise, it can represent that the matching state PM of the first physiological signal PS1 before and after exercise is better, and vice versa.

In practical applications, since the specific index ID can indicate whether the physiological state of the examinee (for example, during exercise, but not limited to this) is normal, the detection device 1 can determine whether the examinee has a specific disease (such as coronary artery disease, but not limited to this) according to the specific index ID.

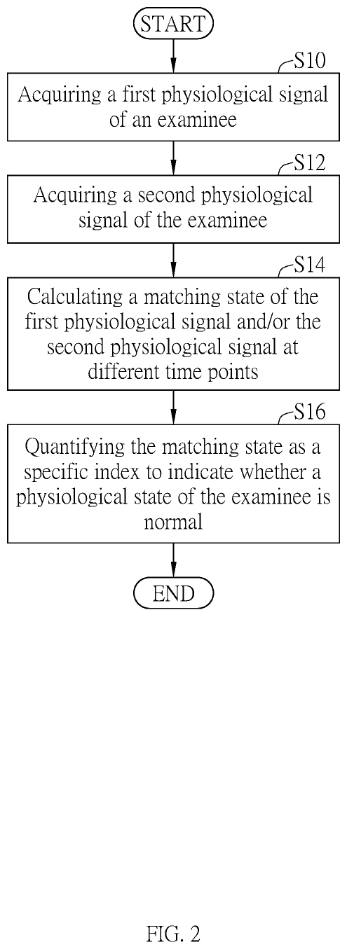

Another embodiment of the invention is a non-invasive method for detecting the coupling of physiological signals. Please refer to FIG. 2. FIG. 2 illustrates a flowchart of the method for detecting the coupling of physiological signals in this embodiment.

As shown in FIG. 2, the detection method includes the following steps:

Step S10: acquiring a first physiological signal of an examinee;

Step S12: acquiring a second physiological signal of an examinee, wherein the second physiological signal is different from the first physiological signal;

Step S14: calculating a matching state of the first physiological signal and/or the second physiological signal at different time points; and

Step S16: Quantifying the matching state as a specific index to indicate whether the physiological state of the examinee is normal.

In practical applications, the examinee in the step S10 and the step S12 can be in an exercise state or other states; the step S14 can calculate the phase match between the first physiological signal and the second physiological signal or other indexes and characteristics, or calculate the phase matching or the matching state of other indexes and characteristics between the first physiological signal and the second physiological signal before and after the measurement (such as waiting before exercise and rest after exercise, but not limited to this); the first physiological signal and the second physiological signal can be related to the heart rhythm information and breathing information of the examinee respectively, but not limited to this.

For example, in the step S10, electrocardiogram (ECG) or photoplethysmogram (PPG) can be used to acquire the heart rhythm information of the examinee during exercise, but not limited to this. In the step S12, a chest strap, a nasal flow detector, or an ECG derived respiration (EDR) algorithm can be used to acquire the breathing information of the examinee during exercise, but not limited to this.

In the step S14, the phase matching or the matching state of other indexes and characteristics can be converted into specific index through Hilbert-Huang transformation (HHT) or other conversion methods; the specific index can be used to determine whether the examinee has a specific disease, such as coronary artery disease, but not limited to this.

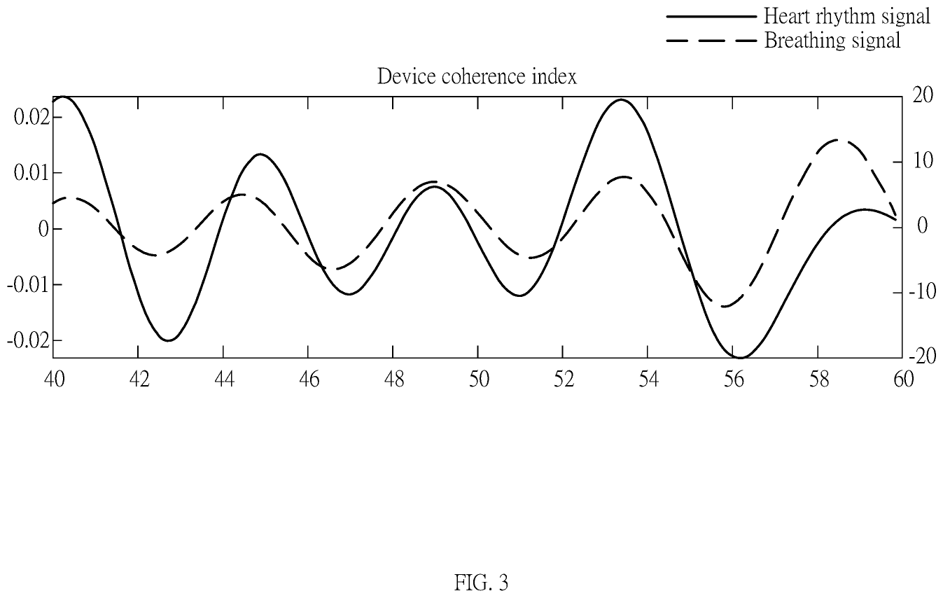

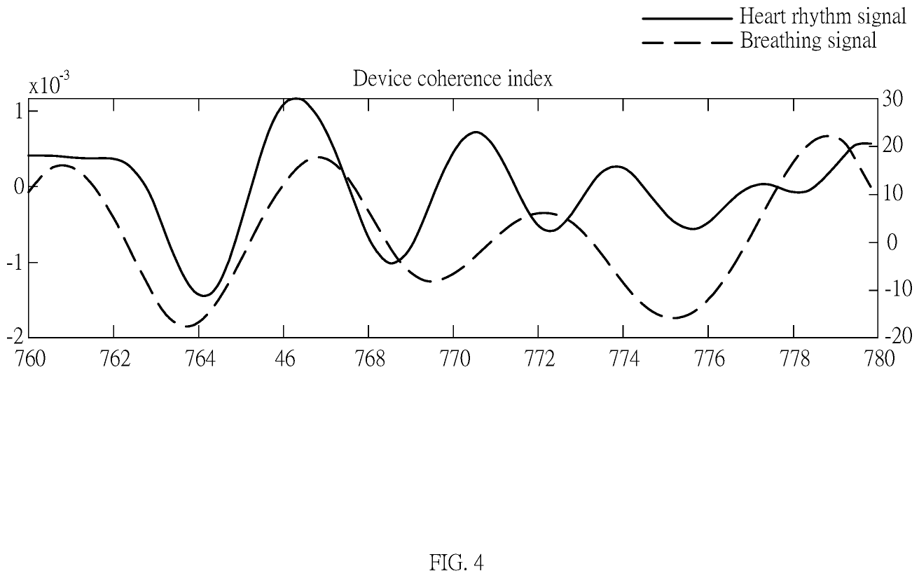

Next, please refer to FIG. 3 and FIG. 4. FIG. 3 illustrates a schematic diagram showing high coupling between the heart rhythm signal and the breathing signal of the examinee during exercise. FIG. 4 illustrates a schematic diagram showing the low coupling between the heart rhythm signal and the breathing signal of the examinee during exercise.

As shown in FIG. 3, the solid line represents the fourth mode with an average frequency of 0.25 Hz after the heart rhythm signal of the examinee during exercise is decomposed, and the dashed line represents the fourth mode with an average frequency of 0.25 Hz after the breathing signal of the examinee during exercise is decomposed. According to FIG. 3, it can be seen that since the coupling between the solid line and the dashed line is high, that is to say, the phase matching state of the two is good. Therefore, when the phase matching state of the two is quantified as a specific index through Hilbert-Huang transform (HHT), it will be determined that the examinee does not have coronary artery disease.

As shown in FIG. 4, the solid line represents the fourth mode with an average frequency of 0.25 Hz after the heart rhythm signal of the examinee during exercise is decomposed, and the dashed line represents the fourth mode with an average frequency of 0.25 Hz after the breathing signal of the examinee during exercise is decomposed. According to FIG. 4, it can be seen that since the coupling between the solid line and the dashed line is low, that is to say, the phase matching state of the two is poor. Therefore, when the phase matching state of the two is quantified as a specific index through Hilbert-Huang transform (HHT), it will be determined that the examinee has coronary artery disease.

Next, the detection results of an embodiment will be used to illustrate how the invention effectively avoids the clinically high false positive rate of the conventional exercise ECG analysis method, so as to improve its accuracy in determining cardiovascular diseases.

In this embodiment, it is assumed that there are fifteen examinees in total, including nine healthy examinees and six examinees with coronary artery disease. If the conventional exercise ECG analysis method is used to determine whether an examinee has coronary artery disease, since all examinees can see a significant decrease in the ST segment on the exercise ECG, all of the nine healthy examinees and the six examinees with coronary artery disease will be determined to have coronary artery disease. This is the clinically high false positive rate (in this case, the false positive rate is even as high as 60%) in conventional exercise ECG.

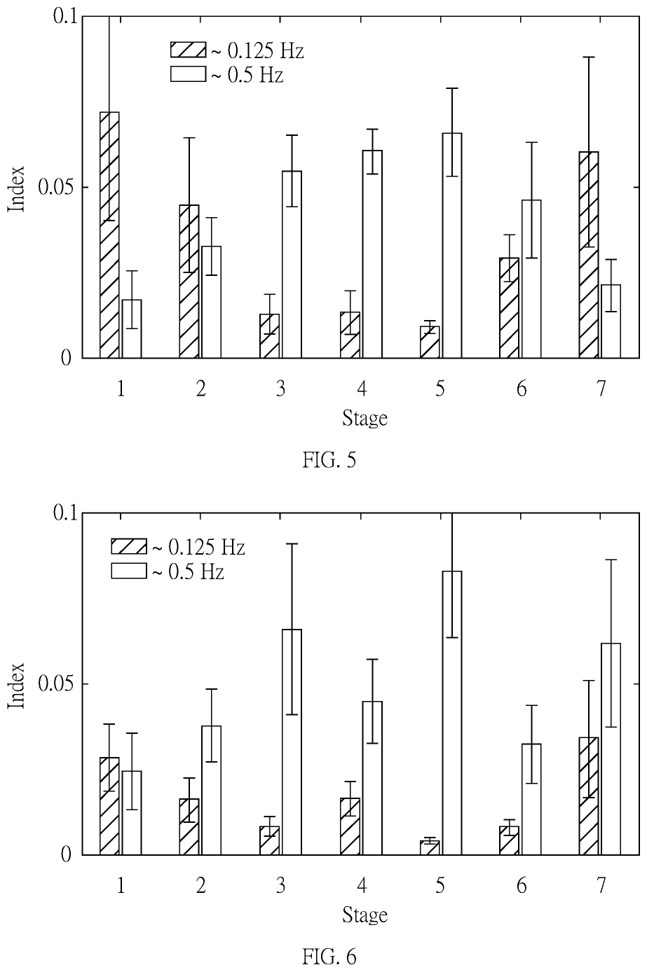

In order to overcome the above-mentioned problems, the device and method for detecting the coupling of physiological signal of the invention firstly collect the heart rhythm signal and the breathing signal of the examinee during exercise, and calculate the coupling (phase matching state) between the heart rhythm signal and the breathing signal and then quantified as a specific index through Hilbert-Huang conversion to clearly distinguish which of the fifteen examinees have coronary artery disease. Please refer to FIG. 5 and FIG. 6 for the test results.

FIG. 5 illustrates a schematic diagram of the change of the index corresponding to the coupling between the heart rhythm signal and the breathing signal of a healthy examinee during exercise in two different modes (frequency 0.5 HZ and 0.125 Hz) over time (stage). FIG. 6 illustrates a schematic diagram of the change of the index corresponding to the coupling between the heart rhythm signal and the breathing signal of an examinee with coronary artery disease during exercise under two different modes (frequency 0.5 HZ and 0.125 Hz) over time (stage).

Even though both healthy examinees and examinees with coronary artery disease showed positive results of coronary artery disease in the exercise ECG test, it can be seen from FIG. 5 and FIG. 6 that there are significant differences in the coupling between the heart rhythm signal and the breathing signal of the examinee with coronary artery disease during exercise. Therefore, the device and method for detecting the coupling of physiological signal of the invention can clearly distinguish the true positive and false positive of coronary artery disease based on the index of the change of the coupling between the heart rhythm signal and the breathing signal of the examinee during exercise. As shown in Table 1 and Table 2, its detection accuracy can reach about 80%.

It should be noted that, in Table 1 and Table 2, numbers 1 to 9 of the examinees represent healthy examinees; numbers 10 to 15 of the examinees represent examinees with coronary artery disease; O represents it is determined that the examinee does not have coronary artery disease; X represents it is determined that the examinee has coronary artery disease.

TABLE 1

| | Number of examinees | | | | | | | | |
|---|---|---|---|---|---|---|---|---|---|
| | 1 | 2 | 3 | 4 | 5 | 6 | 7 | 8 | 9 |
| Actual state | ◯ | ◯ | ◯ | ◯ | ◯ | ◯ | ◯ | ◯ | ◯ |
| Test results of exercise ECG | X | X | X | X | X | X | X | X | X |
| Test results of the invention | ◯ | X | ◯ | ◯ | ◯ | ◯ | ◯ | ◯ | X |

TABLE 2

| | Number of examinees | | | | | |
|---|---|---|---|---|---|---|
| | 10 | 11 | 12 | 13 | 14 | 15 |
| Actual state | X | X | X | X | X | X |
| Test results of exercise ECG | X | X | X | X | X | X |
| Test results of the invention | X | X | X | ◯ | X | X |

According to Table 1 and Table 2, it can be clearly seen that in this embodiment, the detection accuracy of the conventional exercise ECG is only 40% (that is to say, up to 60% of false positives). In contrast, the detection accuracy of the device and method for detecting the coupling of physiological signals of the invention is as high as 80%, which is significantly better than the detection accuracy of the conventional exercise ECG.

Compared to the prior art, the device and method for detecting the coupling of physiological signals of the invention are non-invasive and can quickly determine whether the examinee has cardiovascular disease by collecting the heart rhythm and breathing data of the examinee during exercise, thereby effectively improving the disease determination accuracy of the exercise ECG on cardiovascular disease, so there is no need to further confirm the false positive test results of the exercise ECG, to achieve the substantial effect of saving medical resources and improving medical efficiency and quality.

With the example and explanations above, the characteristics and spirits of the invention will be hopefully well described. Those skilled in the art will readily observe that numerous modifications and alterations of the device may be made while retaining the teaching of the invention. Accordingly, the above disclosure should be construed as limited only by the metes and bounds of the appended claims.

What is claimed is:

1. A device for detecting coupling between physiological signals, comprising:
a first signal acquisition unit, configured to acquire a first physiological signal of an examinee;
a second signal acquisition unit, configured to acquire a second physiological signal of the examinee, wherein the second physiological signal is different from the first physiological signal; and
an operation unit, coupled to the first signal acquisition unit and the second signal acquisition unit respectively and configured to quantify a matching state of the first physiological signal and/or the second physiological signal at different time points as a specific index to indicate whether a physiological state of the examinee is normal.

2. The device of claim 1, wherein the first signal acquisition unit and the second signal acquisition unit acquire the first physiological signal and the second physiological signal of the examinee respectively when the examinee is in an exercise state.

3. The device of claim 1, wherein the operation unit uses Hilbert-Huang transform (HHT) to convert the matching state to the specific index.

4. The device of claim 1, wherein the specific index is used to determine whether the examinee has a specific disease.

5. The device of claim 4, wherein the specific disease is a coronary artery disease.

6. The device of claim 1, wherein the first physiological signal and the second physiological signal are related to a heart rhythm information and a breathing information of the examinee respectively.

7. The device of claim 6, wherein the first signal acquisition unit acquires the heart rhythm information of the examinee through electrocardiography (ECG) or photoplethysmography (PPG).

8. The device of claim 6, wherein the second signal acquisition unit acquires the breathing information of the examinee through a chest strap, a nasal flow detector or an electrocardiography derived respiration (EDR) algorithm.

9. The device of claim 1, wherein the device is a non-invasive detection device.

10. A method for detecting coupling between physiological signals, comprising steps of:
(a) acquiring a first physiological signal of an examinee;
(b) acquiring a second physiological signal of the examinee, wherein the second physiological signal is different from the first physiological signal;
(c) calculating a matching state of the first physiological signal and/or the second physiological signal at different time points; and
(d) quantifying the matching state as a specific index to indicate whether a physiological state of the examinee is normal.

11. The method of claim 10, wherein the examinee in the step (a) and the step (b) is in an exercise state.

12. The method of claim 10, wherein the step (b) converts the matching state to the specific index through Hilbert-Huang transform (HHT).

13. The method of claim 10, wherein the specific index is used to determine whether the examinee has a specific disease.

14. The method of claim 13, wherein the specific disease is a coronary artery disease.

15. The method of claim 10, wherein the first physiological signal and the second physiological signal are related to a heart rhythm information and a breathing information of the examinee respectively.

16. The method of claim 15, wherein the step (a) acquires the heart rhythm information of the examinee through electrocardiography (ECG) or photoplethysmography (PPG).

17. The method of claim 15, wherein the step (b) acquires the breathing information of the examinee through a chest strap, a nasal flow detector or an electrocardiography derived respiration (EDR) algorithm.

18. The method of claim 10, wherein the method is a non-invasive detection method.
